(19) United States
(12) Patent Application Publication
(10) Pub. No.: US 2026/0360096 A1
(43) Pub. Date: Nov. 12, 2026

(54) SYSTEM AND METHOD FOR MULTI USER EXTENDED REALITY INTEGRATION AND COORDINATED CONTROL OF MEDICAL SYSTEMS

(71) Applicant: MEDIVIEW XR, INC, Cleveland, OH (US)

(72) Inventors: John Rauch, Mesa, AZ (US); Peter Nicholas Braido, Wyoming, MN (US); Seyedsina Shirinpour, Minneapolis, MN (US); Cameron Nicholas Mar, Cleveland Heights, OH (US); Adam E. Rakestraw, Findlay, OH (US)

(21) Appl. No.: 19/671,624

(22) Filed: May 8, 2026

Related U.S. Application Data
(60) Provisional application No. 63/802,059, filed on May 8, 2025.

(51) Int. Cl. G06T 19/00; G06F 3/01; G16H 40/67

(52) U.S. Cl. CPC G06T 19/006; G06F 3/011; G06F 3/017; G16H 40/67; G06T 2200/24; G06T 2210/41; G06T 2219/024

(57) ABSTRACT

A system and method for multi-user extended reality (XR) integration of medical systems enables coordinated control of diverse medical equipment in interventional suites. A plurality of XR devices display augmented representations to respective users while a computer system integrates with medical systems through tight coupling, enabling direct data interchange over dedicated interfaces, or loose coupling, enabling video capture and simulated control for legacy systems. The computer system maintains spatial registration between integrated medical systems and a suite coordinate space and a patient coordinate space. A temporal data storage module continuously records procedure execution data, a role-based access control module filters displayed content and system control access based on each user's assigned role, and a state tracking module monitors configurations and operational status of all integrated medical systems, enabling multiple users to simultaneously access role-specific information through individual XR devices.

### CROSS-REFERENCE TO RELATED APPLICATIONS

This application claims the benefit of U.S. Provisional Application No. 63/802,059 filed on May 8, 2025. The entire disclosure of the above application is incorporated herein by reference.

### FIELD

The present technology relates to extended reality systems for use in medical environments, and, more particularly, to multi-user extended reality systems, associated computer systems, methods of use, and related technologies.

### INTRODUCTION

This section provides background information related to the present disclosure which is not necessarily prior art.

Medical procedures conducted in interventional suites demand extraordinary coordination among teams of professionals who must simultaneously monitor, interpret, and act upon information generated by a vast array of specialized systems. In a single interventional suite, a physician may rely on X-ray angiography, CT, MRI, or ultrasound imaging; a nurse monitors patient vitals through patient monitoring equipment and manages medication and device inventory; a technician processes images on a dedicated workstation; and an anesthesiologist tracks hemodynamics and drug delivery, all at once, all in the same room. Each of these systems, including imaging, life support, treatment, navigation, and workflow, operates as a standalone unit with its own displays, input devices, controls, and interaction style.

The result of this arrangement is an environment characterized by profound fragmentation. Data is inherently isolated, merging data is cumbersome, and time is wasted switching between system user interfaces, requiring the user to adapt to different system interaction paradigms for each device. The physician standing at the table may have line of sight to a large central display, but that display is positioned for the doctor standing right next to the patient, while anesthesiology is usually removed and off to the side, sometimes receiving a duplicate of some of the information but not all of it. Nurses, technicians, and fellows must each work from wherever their role places them in the room, often without visibility into the information streams most relevant to their responsibilities at any given moment.

The fragmentation is not merely a matter of physical display placement. It extends to the architecture of the systems themselves. Medical equipment from different manufacturers often operates independently, requiring healthcare providers to interact with multiple separate interfaces and controls, and this fragmentation of information and control systems can impede workflow efficiency and communication between team members during procedures. A physician who needs to correlate live X-ray imaging with hemodynamic waveforms and a pre-operative CT volume must mentally synthesize data presented in incompatible formats, on incompatible displays, governed by incompatible controls.

Spatial complexity adds yet another dimension to the problem. As patients or equipment move during procedures, maintaining accurate spatial relationships between imaging data, physical anatomy, and medical devices becomes increasingly challenging, and medical teams must mentally correlate two-dimensional images with three-dimensional anatomy while coordinating multiple systems that each use different methods of spatial registration. An image acquired at one point in time, with the patient table in one position, may be rendered meaningless for guidance purposes the moment the table moves. There is no mechanism to recall the system state, reestablish spatial context, or allow team members to compare images across different procedural time points without significant manual effort.

The roles of team members compound this challenge further. While physicians need detailed imaging data and treatment system controls, nurses must monitor vitals and inventory, and technicians manage image processing tasks. The physical constraints of certain display systems make it difficult to provide each team member with their specific information needs while maintaining coordination across the entire medical team. No existing framework allows each operator to receive a tailored, role-appropriate view of the suite's information landscape while simultaneously enabling those operators to collaborate fluidly, share findings instantly, and hand off control of systems as the procedure evolves.

Accordingly, there is a continuing need for a system and method that unifies the disparate medical systems within an interventional suite under a single, coordinated interface capable of delivering role-specific information to multiple simultaneous operators, maintaining spatial registration across modalities and time points, and enabling integrated control of both modern and legacy systems throughout the full course of a medical procedure.

### SUMMARY

In concordance with the instant disclosure, a system and method that unifies the disparate medical systems within an interventional suite under a single, coordinated interface capable of delivering role-specific information to multiple simultaneous operators, maintaining spatial registration across modalities and time points, and enabling integrated control of both modern and legacy systems throughout the full course of a medical procedure has surprisingly been discovered.

The present technology includes articles of manufacture, systems, and processes that relate to multi-user extended reality integration and coordinated control of medical systems, including hardware, software, and methods for delivering role-specific information to multiple simultaneous operators within a medical environment, maintaining spatial registration across imaging modalities and procedural time points, and enabling unified interaction with and control of otherwise independent medical systems through an extended reality interface.

In one embodiment, a system for multi-user extended reality integration of medical systems can include an extended reality system comprising multiple extended reality (XR) viewing and sensory devices, a computer system in communication with the extended reality system through a communications network, and one or more medical systems in communication with the computer system. The computer system can include a system integration module, registration management modules, a temporal data storage module, a role-based access control module, and a state tracking module. The computer system can implement tight coupling methodologies enabling direct data interchange with reduced bandwidth, loose coupling methodologies enabling video capture and simulated control interfaces, and can enable multiple local and remote users to simultaneously access role-specific information through individual XR viewing and sensory devices while maintaining registration validity across different modalities.

In another embodiment, a method for multi-user extended reality integration of medical systems can begin by establishing communication between an extended reality system and a computer system through a communications network. The method can implement both tight coupling methodologies enabling direct data interchange with medical systems and loose coupling methodologies enabling video capture and simulated control interfaces through system integration modules. The method can register multiple medical systems with different registration modalities, enable role-based access control to manage information distribution to different users, track system states and configurations using confidence values for each integrated system, and store temporal data about movements, equipment positions, and system states. The method can further enable multiple medical professionals to simultaneously access role-specific information through individual XR viewing and sensory devices while synchronizing system states across users.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic diagram of a system for multi-user extended reality integration and coordinated control of medical systems.

FIG. 2 is a schematic diagram illustrating the network architecture of the system.

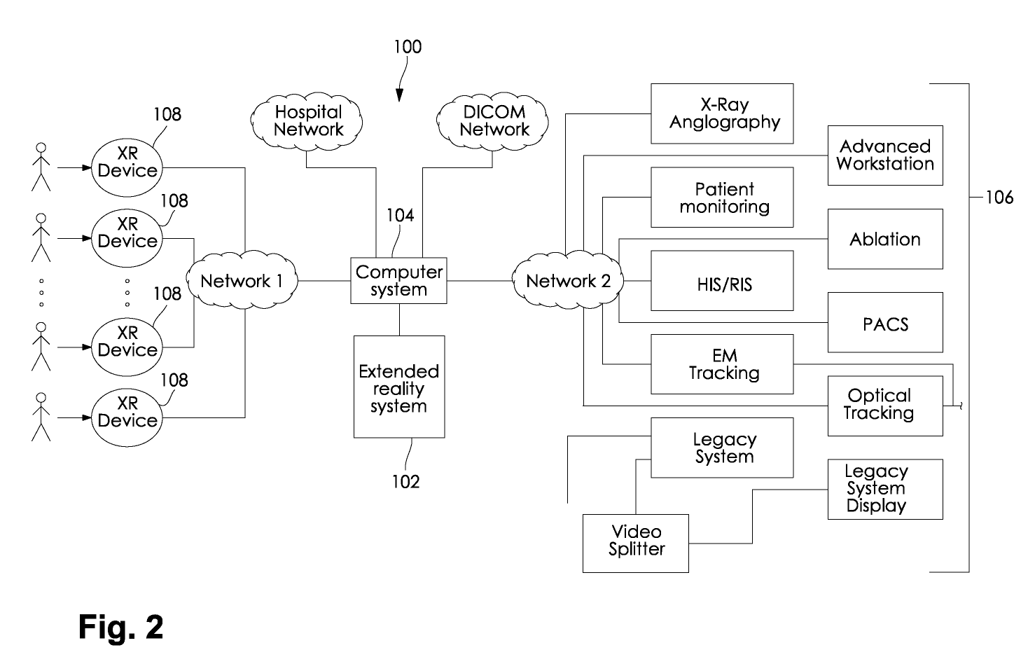

FIG. 3 is a perspective view of a medical procedure environment in which the system is deployed.

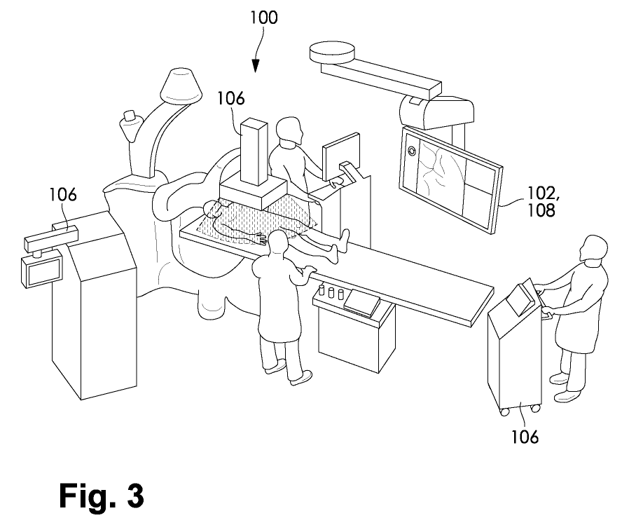

FIG. 4 is a view of a plurality of virtual panels rendered within the extended reality environment during a tight integration scenario.

FIG. 5 is a view of a plurality of virtual panels and a virtual keyboard rendered within the extended reality environment during a loose integration scenario.

FIG. 6 is a flowchart illustrating a method of operating the system for extended reality integration and coordinated control of medical systems.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1–10, or 2–9, or 3–8, it is also envisioned that Parameter X may have other ranges of values including 1–9, 1–8, 1–3, 1–2, 2–10, 2–8, 2–3, 3–10, 3–9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

As used herein, the terms 'interventional device' or 'tracked instrument' refers to any medical instrument used during a medical procedure, including but not limited to needles, probes, scalpels, trocars, screwdrivers, forceps, syringes, and other surgical tools and instruments.

As used herein, the term 'tracking system' refers to something used to observe one or more objects undergoing motion and supply a timely ordered sequence of tracking data (e.g., location data, orientation data, or the like) in a tracking coordinate system for further processing. As an example, the tracking system can include electromagnetic tracking that can observe an interventional device equipped with a tracker, optical tracking that can detect reflective markers or light sources, fiber optic shape sensing for flexible catheters and instruments within the body, electro-anatomical mapping, impedance-based tracking, or other tracking modalities that can observe the interventional device as it enters, moves through and/or in, and exits a patient's body, as well as while outside of the patient's body.

As used herein, the term “tracking data” refers to information recorded by the tracking system related to an observation of one or more objects undergoing motion.

As used herein, the terms “head-mounted device,” “headset,” or “HMD” refer to a display device configured to be worn on the head that has one or more display optics, including lenses, in front of one or more eyes. These terms may be referred to more generally by the term “extended reality” or “XR,” which encompasses extended reality and mixed reality systems, where mixed reality refers to interactive environments that combine physical objects with their related data in a manner similar to Medical Internet of Things (MIoT) applications. XR includes pass-through virtual reality devices. It should be appreciated that the term “extended reality system” is not limited to display devices configured to be worn on the head. In some embodiments, the head-mounted device can also include a non-transitory memory and a processing unit. Examples of suitable head-mounted devices include the Microsoft HoloLens (Microsoft, Redmond, Washington), Magic Leap 2 (Magic Leap, Inc.), and DigiLens (DigiLens Inc.) devices. Additional suitable head-mounted devices can include the XREAL Aura (XREAL Inc.) and pass-through virtual reality headsets such as the Apple Vision Pro (Apple Inc.) and Meta Quest 3 (Meta Platforms, Inc.), among others.

As used herein, the terms “imaging system,” “image acquisition apparatus,” “image acquisition system” or the like refer to technology that creates a visual representation of the interior of a patient’s body. For example, the imaging system can be a computed tomography (CT) system, a fluoroscopy system, positron emission computed tomography, magnetic resonance imaging (MRI) system, an ultrasound (US) system including contrast agents, color flow doppler, 4D ultrasound with volume rendering capabilities, or the like.

As used herein, references to specific imaging modalities such as 'CT' or 'MRI' are intended to be exemplary only. Any reference to a particular imaging modality should be understood to encompass CT, MRI, PET, and other tomographic imaging modalities that can be used interchangeably within the system for visualization, registration, and guidance purposes, unless explicitly stated otherwise.

As used herein, the terms “coordinate system” or “extended reality system coordinate system” or “extended reality system coordinates” refer to a 3D Cartesian coordinate system that uses one or more numbers to determine the position of points or other geometric elements unique to the particular extended reality system or image acquisition system to which it pertains. This includes coordinate data from both internal sensors within the headset (such as inertial measurement units and optical sensors) as well as external sensors that may be attached to the headset and/or user for enhanced tracking capabilities. For example, 3D points in the headset coordinate system can be translated, rotated, scaled, or the like, from a standard 3D Cartesian coordinate system.

As used herein, the terms “image data” or “imaging dataset” or “imaging data” refers to information recorded by the imaging system related to an observation of the interior of the patient’s body. For example, the “image data” or “imaging dataset” can include processed two-dimensional or three-dimensional images or models such as imaging images, e.g., represented by data formatted according to the Digital Imaging and Communications in Medicine (DICOM) standard or other relevant imaging standards.

As used herein, the terms “imaging coordinate system” or “image acquisition system coordinate system” refers to a coordinate system that uses one or more numbers to determine the position of points or other geometric elements unique to the particular imaging system. This includes three-dimensional orthogonal coordinate systems such as Cartesian coordinates, cylindrical coordinates, spherical coordinates, bipolar cylindrical coordinates, bispherical coordinates, confocal ellipsoidal coordinates, and other orthogonal curvilinear coordinate systems, as well as quaternions for representing three-dimensional rotations. For example, points and vectors in the imaging coordinate system can be translated, rotated, scaled, or the like, to the Extended reality system (head mounted displays) coordinate system.

As used herein, the terms “hologram”, “holographic,” “holographic projection”, or “holographic representation” refer to a computer-generated image stereoscopically projected through the lenses of a headset. Generally, a hologram can be generated synthetically (in an extended reality (AR)) and is not a physical entity.

As used herein, the term “physical” refers to something real. Something that is physical is not holographic (or not computer-generated).

As used herein, the term “two-dimensional” or “2D” refers to something represented in two physical dimensions.

As used herein, the term “three-dimensional” or “3D” refers to something represented in three physical dimensions. An element that is “4D” (e.g., 3D plus a time and/or motion dimension) would be encompassed by the definition of three-dimensional or 3D.

As used herein, the term “integrated” can refer to two or more things being linked or coordinated. For example, a coil-sensor can be integrated with an interventional device. As used herein, the term “real-time” or “near-real time” or “live” refers to the actual time during which a process or event occurs. In other words, a real-time event is done live (within milliseconds so that results are available immediately as feedback). For example, a real-time event can be represented within 100 milliseconds of the event occurring.

As referred to herein, the term “sensor fusion” refers to the process of combining data from multiple sensors or sensor modalities to produce a more accurate, reliable, or complete result than could be achieved using any single sensor or modality alone. In the context of tracking, sensor fusion typically combines data from multiple tracking modalities (e.g., electromagnetic, optical, and inertial) to produce position and orientation estimates that compensate for the limitations of individual modalities.

As referred to herein, the term “registration” refers to the process of determining the spatial relationship between two or more coordinate systems. Once registration is established, positions in one coordinate system can be transformed to corresponding positions in another coordinate system. Registration enables virtual content to be displayed in spatially correct locations relative to real-world objects. Registration may be rigid (preserving distances and angles) or non-rigid (allowing for deformation).

As referred to herein, the term “multi-planar reconstruction” or “MPR” refers to a technique for visualizing medical imaging data as two-dimensional slices in arbitrary orientations. While medical images are typically acquired in standard planes (axial, sagittal, coronal), multi-planar reconstruction allows visualization of slices in any orientation, including oblique planes aligned with anatomical structures or interventional devices.

As referred to herein, the term “segmentation” refers to the process of identifying and extracting specific structures from medical images. Segmentation may be manual (user-delineated), semi-automatic (user-guided with automatic processing), or fully automatic (algorithm-based without user intervention). Segmentation produces a three-dimensional representation of anatomical structures such as organs, bones, vessels, or pathological structures such as tumors. The segmented structures can be visualized as holographic overlays.

As referred to herein, the term “fiducial” or “fiducial marker” refers to a marker placed on or attached to a patient to provide a reference point visible in both medical images and the real world. Fiducials may be radiopaque (visible in X-ray, CT, or fluoroscopy), MRI-visible, visible to optical sensors, or detectable by multiple modalities. Fiducials enable registration by providing corresponding points that can be identified in imaging data and in the physical world.

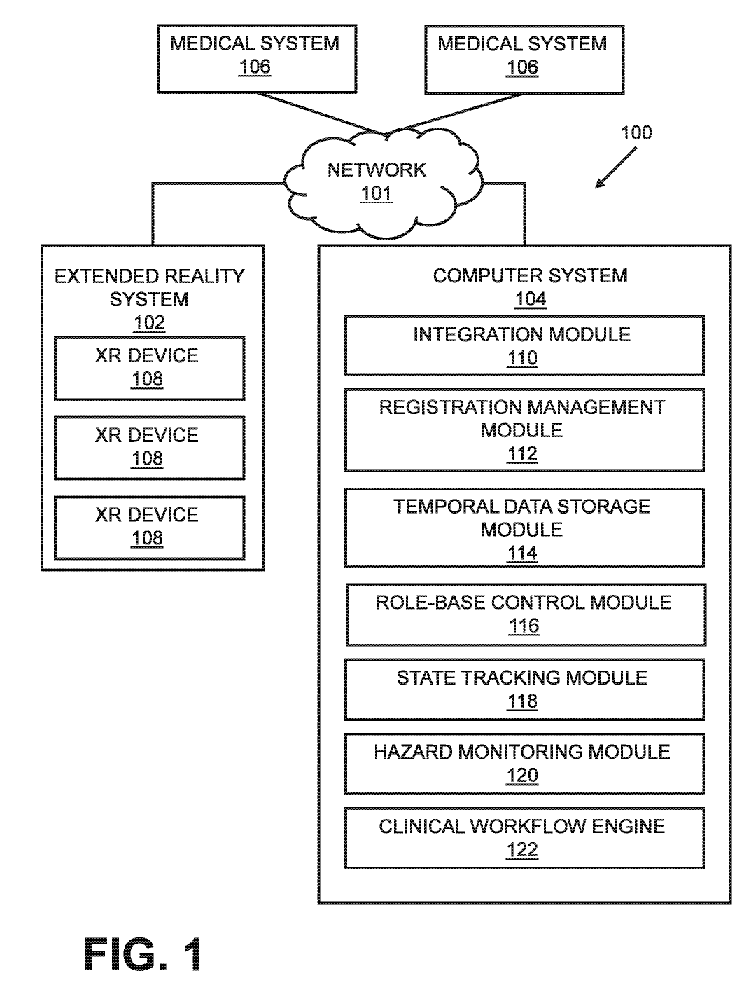

With reference to FIG. 1, a system 100 for multi-user extended reality integration and coordinated control of medical systems can include an extended reality system 102, a computer system 104, and one or more medical systems 106. The computer system 104 can be in communication with the extended reality system 102 through a network 101, which can include various types of communication networks including one or more wide-area networks such as the Internet, local area networks, private and public 5G/6G networks, and other wired and wireless communication links. The medical systems 106 can likewise be in communication with the computer system 104 through the same network 101 infrastructure or through dedicated communication channels, as described in greater detail herein.

The system 100 can be configured to serve as a unified interface layer across an entire interventional suite, replacing the fragmented collection of individual displays, input devices, and control paradigms with a single coordinated extended reality environment accessible to every operator in the room. Rather than relying on a large central display positioned for one primary user, the system 100 can distribute information to every actor in the suite, enabling each operator to see precisely the information they need, when they need it, while retaining the ability to quickly share any view or dataset with any other operator on demand. The system 100 can support up to eight participating operators simultaneously, each with a customized user interface and role-specific defaults, according to one non-limiting embodiment.

The computer system 104 can house a plurality of software modules that collectively enable the integration, visualization, registration, data management, and access control capabilities of the system 100. With reference to FIG. 1, these modules can include a system integration module 110, registration management module 112, a temporal data storage module 114, a role-based access control module 116, a state tracking module 118, a hazard monitoring module 120, and a clinical workflow engine 122. The modules can operate independently or in coordination with one another, and in some embodiments the functions of one or more modules may be combined or distributed across additional sub-modules without departing from the scope of the present technology. The extended reality system 102, computer system 104, software modules, and medical systems 106 are each described in greater detail herein.

The extended reality system 102 can serve as the primary interface layer through which all operators in the interventional suite access, interact with, and control the medical systems 106 integrated into the system 100. The extended reality system 102 can be configured to display augmented representations through one or more XR devices 108 simultaneously, enabling both local and remote users to access information. The extended reality system 102 can replace the fragmented collection of physical displays distributed throughout the suite with a unified, personalized information environment that travels with each operator and adapts to their role, position, and needs throughout the course of a procedure.

The XR devices 108 can include, but are not limited to, headsets, transparent displays, projection systems, heads-up displays (HUDs), mobile devices, tablets, robotic consoles, and haptic feedback devices. In embodiments utilizing head-mounted devices, the XR device 108 can include one or more display optics in front of one or more eyes of the user, with the head-mounted device optionally including a non-transitory memory and a processing unit. Examples of suitable head-mounted devices include the Microsoft HoloLens (Microsoft, Redmond, Washington), Magic Leap 2 (Magic Leap, Inc.), DigiLens (DigiLens Inc.) devices, XREAL Aura (XREAL Inc.), and pass-through virtual reality headsets including the Apple Vision Pro (Apple Inc.) and Meta Quest 3 (Meta Platforms, Inc.), among others. In some embodiments, the XR device 108 can include internal sensors such as inertial measurement units and optical sensors for head pose tracking, as well as external sensors attachable to the headset or user for enhanced tracking capabilities.

In embodiments utilizing transparent or see-through display configurations, the XR device 108 can overlay digital content onto the user's natural field of view without obstructing visibility of the physical environment or the patient. In embodiments utilizing projection-based configurations, augmented content can be projected onto surfaces within the procedure room, enabling display of information in locations accessible to multiple observers simultaneously without requiring each observer to wear a personal device. In embodiments utilizing mobile devices or tablets, the XR device 108 can provide a subset of the extended reality interface optimized for touch-based interaction, enabling access by personnel who are not wearing head-mounted devices. In embodiments utilizing robotic consoles, the XR device 108 can present the extended reality interface through the display system of the console, enabling operators who are physically remote from the patient to access the same integrated suite information environment as operators present in the room. In embodiments utilizing haptic feedback devices, the XR device 108 can provide tactile feedback corresponding to interactions with virtual interface elements, augmenting the visual and auditory interaction modalities available to the user.

The system 100 is not limited to a single XR device type per user or per deployment. In some embodiments, different operators within the same procedure room can utilize different types of XR devices 108 simultaneously, with each device receiving the same underlying data streams filtered and formatted appropriately for that device's display capabilities and the user's role.

The extended reality system 102 can enable multiple medical professionals to simultaneously use XR devices 108, with each user receiving customized displays showing information relevant to their role. In certain embodiments, the system 100 can support eight participating operators simultaneously, each with a customized user interface and role-specific defaults. It should be understood that certain embodiments of the system 100 can support fewer than eight operators or more than eight operators as deemed appropriate for a particular medical procedure. The information presented to each operator can differ both in content and in presentation format, with the role-based access control module 116 governing what each user sees and what level of control each user has over the systems and data presented to them. Some information can be common across all users and shared simultaneously in all XR environments, while other information can be exclusive to particular users based on their roles in the procedure.

The augmented representations displayed through the XR devices 108 can include two-dimensional and three-dimensional depictions of relevant information for medical procedures, including preoperative and intraoperative data such as two-dimensional and three-dimensional imaging data, time-varying signals representing biological processes, patient biographical information, individual treatment statuses, system state information, three-dimensional depictions of anatomical features, and real-time tracking and visualization of medical devices, instruments, and equipment in the operating environment. Anatomical features that can be depicted include organs, portions of organs, tissues, joints, bones, tumors, vasculature, and implants, among others. Three-dimensional data from any system in use in the suite can be registered to and displayed in the real world, displayed in a standalone virtual space, or registered into a single shared space or separated into individual spaces depending on the viewing context and user preference. Video streaming from any system in use in the suite can similarly be registered to and displayed in the real world or displayed in a virtual heads-up display, while signal data can be displayed as scrolling signal data on a dedicated panel.

The extended reality system 102 can be configured to display augmented representations directly over the relevant portion of the patient in the extended reality environment, allowing practitioners to view holographic representations in the same field of view as the patient and the anatomical feature of interest. This can allow augmented representations to be depicted directly over the anatomical feature, providing relevant feedback within the context of the physical position of that feature.

Each operator can independently configure their extended reality display environment, selecting the medical systems 106 and data streams they wish to view and positioning the corresponding virtual panels at locations within their field of view that are conducive to their working position and role. Virtual panels can operate independently of one another, and each can present a system-specific interface through which the operator can access the controls, data, and visualization options associated with that system. System-specific control widgets can enable users to interact with holographic content and system data within dedicated panels, and system-specific voice commands can be paired with hand gestures to enable hands-free control throughout the procedure.

The XR devices 108 can support a plurality of input modalities for interacting with the extended reality environment and with integrated medical systems 106. Voice commands can be issued naturally during the course of a procedure without requiring the user to redirect their attention or physical contact to a separate input device. Hand gesture recognition can enable users to manipulate virtual panels, select controls, and interact with three-dimensional holographic content through natural hand movements. For tightly coupled systems, dedicated voice commands and virtual user interface elements can allow user interactions through the extended reality system 102, including processing, analyzing, annotating, and measuring three-dimensional images, three-dimensional volumes, two-dimensional images, and time-varying signals. Eye tracking, where supported by the XR device 108, can be used to direct attention-based interactions and, in conjunction with head pose data, to automatically direct the field of view of registered optical systems such as robotic exoscopes, as described in the examples herein.

One particular advantage of the extended reality system 102 over fixed physical display arrangements can be the ergonomic flexibility the extended reality system 102 provides to each operator. Each operator can place displays at locations conducive to their working position, enabling a natural, unobstructed working posture throughout the procedure. A physician working at the patient table can position imaging panels and control widgets directly in their forward field of view at a comfortable viewing distance, without requiring any physical rearrangement of the room. An anesthesiologist positioned away from the central imaging area can arrange a personalized display environment oriented toward their working station, showing the hemodynamic monitoring and medication administration records most relevant to their role. A nurse circulating in the suite can access inventory systems, vital signs, and medication records through a display configuration optimized for their movement patterns and positional variability throughout the procedure.

The three-dimensional nature of the extended reality environment can enable rapid understanding of complicated anatomy, as three-dimensional structures can be visualized directly in three-dimensional space rather than requiring mental reconstruction from two-dimensional representations. Voice control and hand gestures can further reduce the physical demands of system interaction, enabling operators to maintain focus on the patient and the procedure rather than on navigating physical interfaces.

Another particularly beneficial capability of the extended reality system 102 can be the enablement of interaction with medical systems 106 that are not conventionally accessible within the sterile field. The system 100 can provide the ability to see and interact with a system not otherwise available in the sterile field without breaking sterility. In interventional suite environments, a physician operating within the sterile field cannot physically interact with equipment or displays located outside that field without compromising sterility, requiring them to either redirect their attention to a non-sterile control interface or to communicate their control needs to another operator. The extended reality system 102 can resolve this constraint by virtualizing the user interfaces of all integrated medical systems 106 within the XR environment, enabling a sterile operator to interact with any integrated system through voice commands, gaze-based controls, and gesture-based interactions that require no physical contact with non-sterile surfaces.

The computer system 104 can serve as the central processing and coordination hub of the system 100, housing the software modules and computing infrastructure through which all integration, registration, data management, access control, and state tracking functions can be performed. The computer system 104 can include one or more processors, non-transitory memory, and associated hardware configured to execute the software modules described herein. The computer system 104 can be integral to the extended reality system 102 or can exist as a standalone component. The system 100 can support a range of physical deployment configurations to accommodate different procedure room environments, hospital infrastructure constraints, and performance requirements.

In a first deployment configuration, the computer system 104 can be located within the procedure room environment as a dedicated local server. In this configuration, computing resources can be distributed across XR device components, such as between a headset and a tethered computing pack, to perform necessary functions. Local deployment can minimize network latency for time-sensitive operations such as real-time image rendering, system control, and registration management, which can be beneficial in procedure environments where millisecond-level response times are clinically meaningful. In a second deployment configuration, the computer system 104 can be implemented as a cloud computing resource or other remote server accessed via the communication network. In cloud-based deployments, the computer system 104 can leverage scalable computing infrastructure to support computationally intensive tasks such as three-dimensional image processing, surface reconstruction from LIDAR point cloud data, and behavioral analytics processing, without requiring dedicated high-performance hardware within the procedure room itself.

In a third deployment configuration, computing resources can be distributed across multiple nodes, including both local and remote components operating in a coordinated manner. In this hybrid configuration, latency-sensitive functions such as XR rendering and real-time system control can be handled by local computing resources, while storage-intensive and computationally intensive functions such as temporal data archiving, DICOM file generation, and analytics processing can be offloaded to remote or cloud-based infrastructure. This distributed approach can allow the system 100 to balance performance, cost, and scalability requirements across diverse deployment environments. In embodiments where the system 100 can be deployed across multiple procedure rooms within the same facility, computing resources can be centralized at a hospital network level and shared across rooms, with appropriate network segmentation and access control protections maintaining separation between the procedure environments of different rooms.

The computer system 104 can include the plurality of software modules that collectively enable the integration, visualization, registration, data management, and access control capabilities of the system 100. The system integration module 110, the registration management module 112, the temporal data storage module 114, the role-based access control module 116, and the state tracking module 118 can work together to provide comprehensive management of procedure information, user access, and system states. The computer system 104 can additionally include the hazard monitoring module 120 and a clinical workflow engine 122, each described in greater detail in the sections that follow. The modules can operate independently or in coordination with one another, and in some embodiments the functions of one or more modules may be combined or distributed across additional sub-modules without departing from the scope of the present technology.

The processing demands of the system 100 can vary substantially across different deployment scenarios depending on the number of concurrently active XR devices 108, the number and types of medical systems 106 integrated, the volume and resolution of imaging data being processed, and the computational complexity of active registration and rendering tasks. The number of headsets and the infrastructure for wireless communication and processing can be scaled together, with larger deployments utilizing different processing hardware while retaining the same fundamental system components and architecture.

The computer system 104 can be configured to prioritize computational resources among competing tasks based on clinical urgency and latency requirements. Real-time rendering and system control functions can be allocated dedicated processing resources to ensure consistent response times regardless of the load imposed by concurrent background processes such as temporal data archiving, analytics computation, or DICOM file generation. In embodiments where the computer system 104 can be implemented as a cloud computing resource, auto-scaling capabilities can allow processing capacity to expand dynamically during periods of peak demand and contract during lower-utilization periods.

The computer system 104 can implement data security and access control protections across all network segments and data storage components. The network infrastructure can implement different access control protections across the various networks to safeguard information and restrict access based on specific user roles. In embodiments where the system 100 can be connected to a hospital information network, the computer system 104 can implement network segmentation to maintain separation between clinical procedure data and administrative systems while still enabling authorized data interchange. Authentication and authorization mechanisms can be implemented at the network, application, and data levels to ensure that access to patient information and system controls can be restricted to appropriately credentialed users. Audit logging capabilities can record user access events, system interactions, and data exchanges throughout the course of procedures, supporting compliance with applicable healthcare data security requirements.

The medical systems 106 with which the system 100 can communicate span the full range of devices utilized in interventional suites and operating theaters. These systems fall within five broad categories: imaging systems, life support and patient monitoring systems, treatment systems, navigation systems, and workflow systems. The system 100 can interface with each category through tight coupling, loose coupling, or a combination of both methodologies, as described in greater detail herein.

The system 100 can be in communication with medical systems 106 including CT, MRI, ultrasound, X-ray angiography systems, and mobile imaging systems such as mobile X-ray, infrared imaging, and optical coherence tomography (OCT) imaging. Additional imaging modalities can include intravascular ultrasound (IVUS), molecular imaging (MI), and scope-based imaging systems. For imaging systems with a fixed spatial relationship to the suite, the system 100 can understand the fixed image space of the imaging system relative to the suite, track all imaging geometry and table movement, and display all two-dimensional and three-dimensional images acquired by the imaging system in their proper spatial relation within the procedure room, with virtual content updated with table movement to stay registered with the patient. For imaging systems with variable imaging geometry, such as X-ray angiography systems utilizing a C-arm, the system 100 can track imaging geometry movements to correctly display images in their relative positions within the extended reality environment.

Through integration with advanced imaging workstations, technicians and other designated users can access workstation functions directly through the extended reality system 102 to perform image analysis, image processing, and procedure planning tasks, generating derived data including detailed anatomical models, target location markers, tumor visualizations, and procedural exclusion zones, which can then be automatically registered and displayed through the extended reality system 102 to provide navigational guidance during procedures.

The system 100 can also integrate with surgical microscope systems to incorporate their high-resolution video feeds into the extended reality environment, enabling real-time visualization of microscope imagery through the XR devices 108, including both standard optical imaging and specialized visualization modes such as fluorescence. Through tight coupling with robotic exoscope systems, the system 100 can enable direct control of the exoscope through the XR devices 108, and when the XR device and robotic exoscope are registered together, the system 100 can automatically direct the exoscope's field of view based on the user's head pose and eye tracking data.

The system 100 can interface with patient monitoring equipment and biological signal monitoring systems for tracking vital signs, hemodynamics data, electroencephalogram (EEG) signals, electrophysiology recordings, and other time-varying biological signals, enabling nurses and anesthesiologists to monitor critical parameters through the XR devices 108. Life support systems with which the system 100 can interface can include analyzers, respirators, and drug delivery systems. The system 100 can additionally interface with pharmacology systems for medication management and with power injectors for setting and monitoring injection protocols.

For treatment systems, the system 100 can interface with robotic surgical systems, ablation systems using various energy sources including radiofrequency, cryo, microwave, and laser, stimulator systems, device deployment systems, and infusion devices including drug delivery systems and autoinjectors. The system 100 can also interface with biological analysis and diagnostic systems including blood analyzers, and bio-material removal systems. Through tight coupling with treatment systems, users can access dedicated virtual control interfaces within the extended reality environment that expose the configuration, activation, and monitoring functions of each integrated treatment device.

Navigation systems with which the system 100 can interface can include mapping systems, robotic surgery systems, and tracking systems utilizing electromagnetic and optical tracking modalities. Additional tracking modalities can include fiber optic shape sensing for flexible catheters and instruments, electro-anatomical mapping, and impedance-based tracking. The system 100 can also interface with electro-anatomic mapping systems, allowing physicians to control these systems while maintaining coordinated visualization.

Through integration with hospital information systems, the system 100 can interface with scheduling systems for procedure planning, history systems for accessing patient records, and DICOM and PACS systems for retrieving and archiving stored imaging studies. Additional workflow systems can include hospital information systems (HIS), radiology information systems (RIS), inventory management systems, billing systems, reporting systems, and work list management systems. The system 100 can generate and store DICOM files containing procedure data, coordinated user actions, and workflow information from integrated systems, and can connect with inventory management systems to track surgical supplies and equipment usage. Any operator can gather a list of scheduled procedures, claim an existing procedure, or create a new procedure and enter patient and procedure data, with the system 100 communicating that patient and procedure data to all integrated systems upon procedure start.

The system 100 can additionally interface with environmental control systems such as lighting. The system 100 can also support mobile systems deployed between different procedure rooms, and can provide help and training materials for integrated systems, enabling operators to access system-specific guidance within the extended reality environment during procedures. Communication systems within the suite can likewise be integrated, enabling voice, video, and text-based communication channels to be accessible through the extended reality environment.

The system 100 can interface with radiation monitoring systems through both dedicated radiation monitoring equipment and tight integration with radiation-based imaging systems, to display numeric patient, physician, and staff dosages with warning thresholds and history. For magnetic resonance systems, the system 100 can visualize active magnetic field volumes and gradients through tight coupling, providing user-configurable, role-specific alerts for magnetic field strength thresholds.

The system integration module 110 can implement and manage the communication channels through which the computer system 104 exchanges data and control with the medical systems 106 integrated into the system 100. The system integration module 110 can manage the data interchange between systems, ensuring proper implementation of both tight and loose coupling methodologies while maintaining system-specific features and controls. The system 100 can implement both tight and loose coupling simultaneously across different medical systems 106. For example, an imaging system 106 might be tightly coupled for direct data interchange and registration, while a legacy monitoring device is integrated through loose coupling with video capture and simulated controls. The system integration module 110 can maintain separate communication pathways for tightly integrated systems and loosely integrated systems, coordinating data flow from both categories through a common network infrastructure.

The system integration module 110 selects between tight and loose coupling based on the technical capabilities of each medical system 106. Where a medical system 106 exposes a native data interface, such as a proprietary API, dedicated wired or wireless communication protocol, or Bluetooth interface, the system integration module 110 employs tight coupling to enable direct data interchange at lower bandwidth, lower latency, and higher fidelity. Where a medical system 106 does not expose such a native interface, such as legacy or third-party devices that provide only display output without programmatic access, the system integration module 110 employs loose coupling through video capture and simulated control inputs. This selection can be performed automatically by the system integration module 110 upon device discovery, or can be configured manually by an operator. Because tight and loose coupling methodologies operate over fundamentally different communication pathways and network types, the computer system 104 is configured to normalize and align data received from each coupled medical system 106 into a unified data stream for presentation within the extended reality environment, enabling coherent integration of medical systems 106 regardless of the coupling methodology employed.

Through tight integration, the system 100 can establish direct communication with medical equipment over dedicated wired, wireless, and Bluetooth interfaces that enable lower bandwidth requirements, lower latency, and higher fidelity data exchange. In a tight integration, the two systems communicate over an interface where data is interchanged directly, enabling more efficacious transfer in terms of lower bandwidth, higher fidelity, and quicker response time.

In tight integration scenarios, the extended reality system 102 can interchange data directly between systems, allowing for more efficient transfer of two-dimensional images, three-dimensional images, time-varying signal data such as hemodynamics, system state information, and report-type data with text and graphics. When the location and geometry of the imaging system 106 is known, the system 100 can automatically position acquired images and derived data within the virtual space of the extended reality system 102.

Tight coupling also enables tracking of patient and patient table movements, allowing images acquired of the patient in one position to be fixed relative to the patient and to move appropriately in the virtual space with movement of the patient. The tight coupling enables control over integrated systems, including configuring and initiating actuated movements of patient tables and controlling imaging geometry movements, acquisition settings, and collimation within safety constraints.

For tightly coupled systems, the tight coupling provides dedicated voice commands and virtual user interface elements for controlling integrated systems directly and manipulating provided data, including processing, analyzing, annotating, and measuring three-dimensional images, three-dimensional volumes, two-dimensional images, and time-varying signals, that allow user interactions through the extended reality system 102.

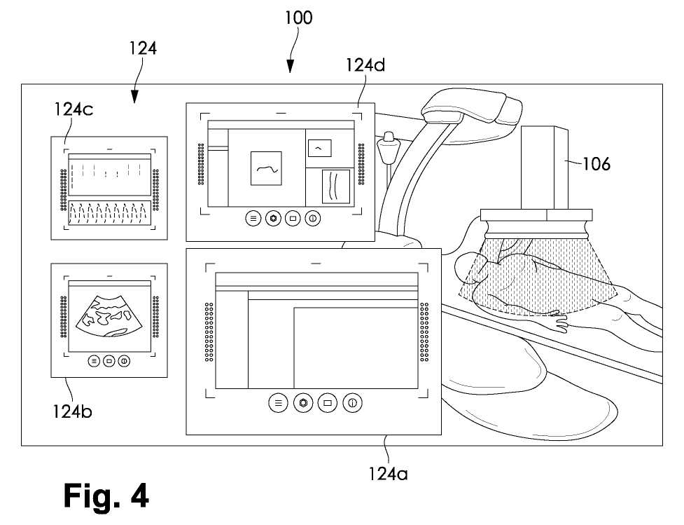

With reference to FIG. 4, in a tight integration scenario the extended reality system 102 can render a plurality of virtual panels 124 within the extended reality environment, each corresponding to a tightly coupled medical system 106. In the illustrated embodiment, four virtual panels 124a, 124b, 124c, and 124d are displayed, though the number and arrangement of virtual panels 124 can vary depending on the systems integrated and the needs of the procedure. Each virtual panel 124a–124d can present data or controls associated with a respective tightly coupled medical system 106, such as two-dimensional or three-dimensional imaging data, time-varying signal data such as hemodynamic waveforms, registration state and tracking information, or system control interfaces for configuring acquisition settings, adjusting collimation, or initiating actuated movements. Each virtual panel 124a–124d is rendered and positioned by the computer system 104 in coordination with the extended reality system 102 and is interactive, allowing users to manipulate, annotate, and control the corresponding medical system 106 through the extended reality interface.

For loose integration scenarios, the computer system 104 can capture video from medical systems 106, either as a separate output or as an interrupted capture, and replicate that video onto virtual panels in the extended reality system 102. This approach enables the system 100 to surface the outputs of legacy or third-party systems within the shared extended reality environment without requiring a dedicated integration interface from those systems.

The system 100 can implement interactions with loosely coupled systems by converting gesture commands on the displayed video panels into emulated mouse cursor positions and clicks, allowing users of the extended reality system 102 to control legacy systems through simulated mouse movement and button clicks. The loose coupling methodology also supports text input through various means, including virtual keyboard implementations and voice dictation with optional auto-correction for unknown words.

The system integration module 110 can include a video capture component, a video splitter component, and a mouse emulator component for implementing loose coupling with legacy systems, with the captured video replicated to both the legacy system display and virtual panels within the extended reality environment.

The system 100 can implement both tight and loose coupling simultaneously across different medical systems 106, allowing the system 100 to maximize integration capabilities based on each medical system's technical capabilities while maintaining coordinated visualization and control throughout medical procedures. In a tightly coupled integration with an external system, the dedicated virtual user interface will be designed jointly by both parties for the benefit of the user. In a loosely coupled situation where all the system is doing is taking video from a system and simulating UI input to it, the panel presents the video with augmented UI controls for mouse and keyboard interaction.

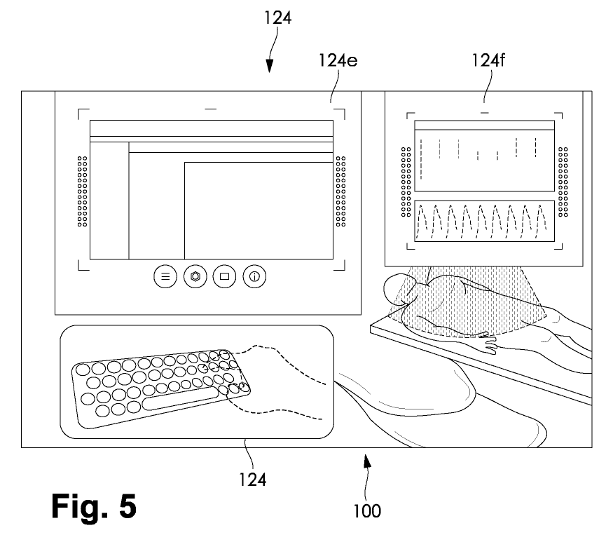

With reference to FIG. 5, in a loose integration scenario the extended reality system 102 can render a plurality of virtual panels 124 within the extended reality environment, each presenting a captured video output from a respective loosely coupled medical system 106. In the illustrated embodiment, two virtual panels 124e and 124f are displayed, though the number and arrangement of virtual panels 124 can vary depending on the systems integrated and the needs of the procedure. Each virtual panel 124e and 124f can present the display output of a respective loosely coupled medical system 106, such as an imaging device, a patient monitoring system, a legacy clinical workstation, or a third-party device providing only display output without a native programmatic interface. Users can interact with each loosely coupled medical system 106 through the corresponding virtual panel 124 by performing gestures that are converted by the computer system 104 into emulated mouse cursor positions and clicks. The extended reality system 102 can also render a virtual keyboard 126 enabling text input to loosely coupled medical systems 106, including through virtual keyboard interaction or voice dictation with optional auto-correction, without requiring a dedicated integration interface from those systems.

The registration management module 112 can maintain spatial relationships for both tightly and loosely coupled systems, though with different levels of precision based on the integration method. For systems utilizing tight coupling methodologies, the registration management module 112 can establish direct communication over dedicated interfaces that enable lower bandwidth requirements and higher fidelity data exchange. For loosely coupled systems, the registration management module 112 can support video capture-based registration and enable simulated user interface inputs through gesture commands and virtual controls.

The system integration module 110 can communicate with medical systems 106 across two distinct network segments: a first network connecting the computer system 104 to tightly integrated systems including imaging systems, patient monitoring equipment, advanced workstations, and electromagnetic and optical tracking systems, and a second network connecting the computer system 104 to hospital systems including HIS/RIS and PACS. In embodiments where the system 100 is connected to a broader hospital network, the computer system 104 can implement network segmentation to maintain separation between clinical procedure data and administrative hospital information systems while still enabling authorized data interchange between these environments.

The registration management module 112 can understand and maintain the different registration modalities used by each medical system 106 integrated into the system 100. An advantage of the system 100 is the ability to understand and maintain different registration modalities across integrated medical systems, while also enabling unregistered simultaneous viewing of disparate data items side by side in separate displays. The registration management module 112 can understand the unique data space and coordinate system for each medical system 106, enabling the system 100 to perform data space transformations that allow information from one system to be displayed relative to data from other systems within the extended reality environment.

The registration management module 112 can support multiple coordinate spaces, including a suite coordinate space, a patient coordinate space, and system-specific coordinate spaces. The user can register among coordinate spaces and can display data from any system in any coordinate space to which the source system is registered. The suite coordinate space can represent the physical coordinate space of the procedure room, detected by the extended reality system 102 through virtual space tracking and registered to the extended reality system 102 by definition of a key landmark. For imaging systems with a fixed spatial relationship to the suite, the system 100 can understand the fixed image space of the imaging system relative to the suite, track all imaging geometry and table movement, and display all two-dimensional and three-dimensional images acquired by the imaging system in their proper spatial relation within the room, with virtual content updated with table movement to stay registered with the patient. The patient coordinate space can define the spatial relationship between acquired imaging data and the patient's physical anatomy, enabling virtual content to remain registered to the patient as the patient or patient table moves. The system-specific coordinate spaces can capture the unique data representations and coordinate conventions of each integrated medical system 106, which the registration management module 112 can transform into the common suite or patient coordinate space for unified display.

Different medical systems 106 can employ different approaches to spatial registration. An imaging system 106 may use physical markers for registration, optical tracking, or non-contact registration methods such as computer vision or surface matching algorithms. A treatment delivery system might use electromagnetic tracking, while a patient monitoring system may register based on fixed room coordinates. The registration management module 112 can maintain awareness of each system's registration approach and apply the appropriate coordinate transformation to align data from that system with the shared extended reality environment.

Tracking modalities supported by the registration management module 112 can include electromagnetic tracking, optical tracking, fiber optic shape sensing for flexible catheters and instruments, electro-anatomical mapping, and impedance-based tracking, among others. The registration management module 112 can additionally support LIDAR-based registration. Through integration with LIDAR sensors in XR devices or standalone scanners, the system 100 can perform automatic registration by capturing and analyzing the surface topology of both patients and equipment. The registration management module 112 can process LIDAR point cloud data to generate comprehensive surface maps, enabling automatic registration without requiring manual placement of fiducial markers. For patient registration, the system 100 can analyze LIDAR-captured surface contours and use statistical shape modeling to automatically identify anatomical landmarks and generate scaling factors. When combined with ultrasound volume sweeps for subsurface registration, this multi-modal approach enables comprehensive registration both at the surface level through LIDAR and below the surface through reconstructed ultrasound volumes.

Through the registration management module 112, disparate data elements from different registered systems can be displayed registered to each other, including one or more two-dimensional images displayed in-situ in relation to one or more acquired three-dimensional images, multiple two-dimensional images from separate systems displayed in-situ to each other, and one or more acquired three-dimensional images from separate systems displayed in-situ to each other. This multi-modal registration capability provides significant value since each imaging modality has unique strengths and weaknesses in depicting different structures or biological processes, and combining these images directly relieves users from the burden of interpreting them separately and merging them mentally.

The registration management module 112 can track the registration state of each integrated system using system-specific confidence values, accuracy values, and precision values, allowing the system 100 to maintain coordinated visualization even when different systems use different approaches to spatial registration. The state tracking module 118 works with the registration management module 112 to maintain confidence values for registrations that are specific to each integrated system. When tracking registration validity, the system 100 can compute and display spatial constraints for user head pose and orientation in real-time, which depend on the position of the user, orientation, distance to observed anatomy, and characteristics of various integrated sensors including depth cameras, infrared sensors, acoustic sensors, and other tracking modalities.

The registration management module 112 can work in conjunction with patient table tracking and patient movement monitoring systems to automatically adjust the positioning of virtual displays and content as movement occurs. The registration management module 112 may interface with separate optical or infrared tracking systems for monitoring patient movements and apply additional registration transformations to preserve spatial relationships of acquired data directly to the patient's anatomy. When the patient table or other equipment moves, the registration management module 112 can store and synchronize temporal data about the movement across different system times for tightly integrated systems, enabling users to reset system states to visualize how positions have changed while maintaining registration validity across different time points. When the user needs to recall an image that was stored during the procedure, the system 100 can reset the system state and show how things have moved, allowing the physician to compare information from different points in time, for example, the state of the system before and after a given movement, while maintaining the validity of the registration at each respective time point.

The registration management module 112 can maintain spatial relationships for both tightly and loosely coupled medical systems 106, though with different levels of precision based on the integration method. For systems utilizing tight coupling methodologies, the registration management module 112 can establish direct communication over dedicated interfaces that enable lower bandwidth requirements and higher fidelity data exchange. For loosely coupled systems, the registration management module 112 can support video capture-based registration and enable simulated user interface inputs through gesture commands and virtual controls.

The temporal data storage module 114 can provide the system 100 with the ability to continuously record comprehensive procedure execution data throughout the course of a medical procedure and to enable users to later retrieve and review system states from any prior point in time while maintaining registration validity. During medical procedures, the temporal data storage module 114 can continuously store comprehensive procedure execution data including user positions and interactions, stored data such as images, signals, and analyses, system states including imaging geometry, patient table geometry, and patient geometry, and administered treatments, with associated timestamps. When changes occur in acquired data, such as new images, signals, treatment delivery, or patient table adjustments, the temporal data storage module 114 can capture this information through direct storage or reference identifiers such as DICOM UIDs, while the state tracking module 118 preserves the contextual system state information. The temporal data storage module 114 can also store key event information not captured by integrated systems, such as treatment device configurations, timing, and duration. Key events can include procedure start and end times, medication administration, treatment delivery, analysis results, and user-defined events for tracking procedure execution and workflow efficiencies.

When the user needs to recall an image that was stored during the procedure, the system 100 can reset the system state and show how things have moved, allowing the physician to compare information from different points in time, for example, the state of the system before and after a given movement, while maintaining the validity of the registration at each respective time point. This capability allows the system 100 to present information not merely as a current running state but as a temporally indexed record from which any prior point in time during the procedure can be reconstructed and reviewed. The temporal data storage module 114 can work in coordination with the state tracking module 118 and the registration management module 112 to enable this recall capability. The temporal data storage module 114 retrieves the stored procedure information, the state tracking module 118 reconstructs the system configurations and states from the relevant time point, and the registration management module 112 maintains the registration validity applicable to that time point.

The temporal data storage module 114 can preserve the full context of system configurations at each imaging position, enabling users to quickly recall and restore optimal imaging positions while ensuring consistent spatial registration of all acquired data. For example, during a procedure a user may position the patient table and imaging geometry to obtain optimized imaging of a particular anatomical region, with the system storing this geometric configuration through the temporal data storage module 114. The user can then reposition the table and imaging geometry to optimize visualization of a different anatomical region for treatment delivery, with this second configuration also being stored. After performing treatment at the second position, the system can enable review of imaging from the first position while maintaining proper spatial relationships adjusted for the table and imaging geometry changes.

When post-treatment imaging is desired at the initial imaging position, the system can automatically restore equipment positions through direct control interfaces, communicating with patient table control systems to drive table movement and with imaging systems to adjust imaging geometry to precisely match the stored positional state, enabling acquisition of directly comparable pre- and post-treatment images while maintaining registration validity throughout the procedure.

The temporal data storage module 114 can maintain historical radiation exposure records for each individual, enabling tracking of cumulative doses over multiple procedures. The computer system 104 can compute and present radiation dose estimates for patients, physicians, and other actors in terms of accumulated dose over the course of procedures through the temporal data storage module 114, with accumulated dose for specific anatomic regions determined based on proximity to radiation sources through tracking of user locations and orientations.

Together with the state tracking module 118 and role-based access control module 116, the temporal data storage module 114 can enable automatic documentation of surgical procedures through object identification and tracking, monitoring of sterilization counts, equipment movement, usage time, and other surgical item-specific types of use or misuse. The system 100 can generate and store DICOM files containing procedure data, coordinated user actions, and workflow information from integrated systems.

The temporal data storage module 114 can operate in close coordination with the role-based access control module 116, the state tracking module 118, and the registration management module 112 throughout the procedure. As different medical professionals access historical data through the temporal data storage module 114, the role-based access control module 116 filters the information displayed; nurses can view stored vitals and inventory data, while physicians may access additional treatment system controls and medication information. The state tracking module 118 enables these role-specific workflows by maintaining awareness of what information each user needs at different procedure stages, and works with the registration management module 112 to maintain confidence values for registrations that are specific to each integrated system throughout the temporal record.

The role-based access control module 116 can manage information distribution to each operator based on their assigned role, filtering both the content displayed through each operator's XR device 108 and the level of system control available to that operator. The role-based access control module 116 can enable different virtual guidance to multiple individuals using respective visualization devices, with guidance tailored to the roles of the individual in the operating room.

The role-based access control module 116 filters information display based on user roles, for example, nurses can view stored vitals and inventory data, while physicians may access additional treatment system controls and medication information. Every operator in the room (e.g., nurses, technicians, anesthesiologists, physicians, fellows) can have XR devices 108 and can receive whatever information is pertinent to them at any given time. The nurse can be looking at vitals, patient monitoring, drug inventory, and available devices. The technician can be dealing with image processing. The anesthesiologist can focus on what they are looking at but can also quickly see whatever the physician needs them to see. This distributes data while also restricting it so that each operator sees only what they need to see at any given time. Example role-specific interfaces include a primary physician interface providing access to imaging controls, hemodynamics data, and treatment system controls; an anesthesiologist interface presenting ECG, vital signs, and medication administration records; and a nurse interface presenting vitals, lab results, patient history, and medication records.

The role-based access control module 116 can implement varying levels of active participation, where physicians may have particular levels of control over displayed elements while nurses or assistants have different levels of control. The system 100 can support both active and passive participants, where active participants may have control over virtual content and view sharing, while passive participants can view presentations without control capabilities. Some mixed reality information may be common for all participants, while other information may be specific and only presented to particular users based on their roles in the medical procedure. The medication inventory system can be configured so that technicians do not have access to it while nurses do, in terms of control. The role-based access control module 116 can manage access to microscope controls and visualization settings based on user roles, and for workstation integration can manage appropriate levels of workstation access and control — technicians may have full access to image processing tools, while physicians can provide input and approval through view-only or limited interaction modes.

Each operator can have their own set of displays tailored to their needs, and can place displays at locations conducive to their working position. Each operator can pull up whatever systems they want to see and arrange their display to put the systems wherever they want. The panels are all independent and individually workable, and each has its own unique interface that the user can access. The role-based access control module 116 can enable user-controlled states for individual users, where each user can establish what information they need at a given time and transition to different information as their needs change throughout the procedure.

For system state synchronization, the system 100 can maintain temporal and spatial reference systems that are shared between users through data exchange channels, allowing for conversion between volume frames and times, defining phase loops and time stamps, and ensuring that virtual objects remain properly positioned relative to physical space across multiple views. The system 100 can also synchronize corresponding user interface elements between users, including values of UI elements, selected frame identifiers, display settings, and three-dimensional mouse positions. When changes occur to system states or virtual content positioning, these updates can be synchronized across all connected users while maintaining appropriate role-based access controls.

When users review historical data, the temporal data storage module 114, state tracking module 118, and role-based access control module 116 coordinate to provide a comprehensive view: the temporal data storage module 114 retrieves stored procedure information, the state tracking module 118 reconstructs system configurations and states from that time point, and the role-based access control module 116 ensures users only see information appropriate to their role. Together, these modules enable advanced features including automatic documentation of surgical procedures through object identification and tracking, monitoring of sterilization counts, equipment movement, usage time, and other surgical item-specific types of use or misuse.

The state tracking module 118 can monitor the configurations and operational status of all medical systems 106 integrated into the system 100 throughout the course of a medical procedure. The state tracking module 118 monitors the configurations and status of all integrated medical systems using specific confidence values, accuracy values, and precision values. By maintaining continuous awareness of equipment states and operational parameters, the state tracking module 118 enables the system 100 to provide users with contextually accurate information at each stage of the procedure and to coordinate the activities of the other software modules described herein.

When changes occur in acquired data, such as new images, signals, treatment delivery, or patient table adjustments, the temporal data storage module 114 captures this information through direct storage or reference identifiers such as DICOM UIDs, while the state tracking module 118 preserves the contextual system state information. This division of responsibility allows the temporal data storage module 114 to focus on data capture while the state tracking module 118 maintains the broader context in which that data was acquired, including the configuration of imaging systems, the position of the patient table, and the operational state of treatment devices. The state tracking module 118 maintains specific confidence values, accuracy values, and precision values for all equipment positions and movements, enabling users to quickly recall and restore optimal imaging positions while ensuring consistent spatial registration of all acquired data.

The state tracking module 118 enables role-specific workflows by maintaining awareness of what information each user needs at different procedure stages. As the procedure advances through its clinical phases, from access and navigation through diagnosis, treatment planning, treatment delivery, and treatment assessment, the state tracking module 118 can track the current procedure stage and coordinate with the role-based access control module 116 to ensure that each operator's display environment reflects the information most relevant to their role at that point in the procedure. Each clinical task defines an overall system state that includes the configuration and state of each system in the suite and the holographic user interface for each operator, including displays and tools.

The state tracking module 118 works with the registration management module 112 to maintain confidence values for registrations that are specific to each integrated system. When tracking registration validity, the system 100 can compute and display spatial constraints for user head pose and orientation in real-time, which depend on the position of the user, orientation, distance to observed anatomy, and characteristics of various integrated sensors including depth cameras, infrared sensors, acoustic sensors, and other tracking modalities. The state tracking module 118 enables comprehensive tracking and visualization of environmental conditions such as radiation exposure, allowing actors in the environment to become aware of actions during procedures that increase their overall exposure and dose accumulation through the extended reality system 102, enabling real-time adjustment of positioning and procedures to minimize radiation exposure while maintaining procedural effectiveness.

Together, the temporal data storage module 114, role-based access control module 116, state tracking module 118, and system integration module 110 work together to provide comprehensive management of procedure information, user access, and system states, enabling advanced features including automatic documentation of surgical procedures through object identification and tracking, monitoring of sterilization counts, equipment movement, usage time, and other surgical item-specific types of use or misuse.

The hazard monitoring module 120 can track environmental hazards present in the procedure room and deliver real-time hazard information to operators through the extended reality system 102 in a role-specific manner. The computer system 104 includes a hazard monitoring module 120 for tracking environmental hazards such as radiation and magnetic fields from all sources in the procedure room. For radiation monitoring, the hazard monitoring module 120 can collect radiation dosage accumulation for imaging radiation from the imaging system with each image acquisition and collect radiation dosage accumulation for radioactive agents, such as pellets or injectables from the radiation measurement system. The system 100 accounts for three types of shielding: fixed shielding that can be directly accounted for in calculations, mobile shielding like walls and ceiling-mounted shields that can be tracked using the extended reality system 102, cameras, or separate tracking systems, and personal protective equipment that can be detected or automatically applied based on individual actor roles through the role-based access control module 116. When radiation is active from a tightly coupled imaging system, the hazard monitoring module 120 can calculate estimated radiation scatter and evaluate impact on patients, users, and environment without requiring a separate radiation monitoring system.

Individual XR devices 108 of the extended reality system 102 can display immediate exposure risk as holographic color or contour overlays showing regions where radiation is at specific levels, providing visualization features for radiation data from both tightly coupled imaging systems and dedicated radiation measurement systems. The extended reality system 102 can generate both two-dimensional surface radiation gradients projected directly onto individuals in real-time and three-dimensional volumetric radiation gradients displayed on virtual models. For patients specifically, the computer system 104 can track and display dose per anatomy or anatomical region through the extended reality system 102, which is particularly valuable for patients who are susceptible to radiation, have substantial prior radiation exposure, or are undergoing procedures that will utilize significant radiation. Using basic biometric information input for each actor, the computer system 104 can create idealized models to record accumulated dose for specific areas based on proximity to radiation sources through the temporal data storage module 114.

The computer system 104 can compute and present radiation dose estimates for patients, physicians, and other actors in terms of accumulated dose over the course of procedures through the temporal data storage module 114. Through tracking of user locations and orientations using optical or thermal camera systems of the extended reality system 102, the computer system 104 can determine accumulated dose for specific anatomic regions based on proximity to radiation sources, displayed as a color map overlaid on actors in the AR experiences of other users or on idealized models accessible for review.

The system 100 maintains documentation of radiation exposure to individual users through the temporal data storage module 114, though this is designed to be adjunctive to personal dosimetry monitoring devices rather than replacing them. The hazard monitoring module 120 provides user-configurable, role-specific alerts when users move into areas with active radiation doses exceeding defined thresholds. Through the role-based access control module 116, alert thresholds and notification preferences can be configured for different operator roles, calibrating hazard warnings to the exposure standards applicable to each user type.

For magnetic resonance systems, the hazard monitoring module 120 can visualize active magnetic field volumes and gradients through tight coupling, providing user-configurable, role-specific alerts for magnetic field strength thresholds. The magnetic field visualization capability can operate within the same holographic overlay framework as the radiation visualization system, enabling operators to view three-dimensional representations of magnetic field boundaries within the procedure room environment.

The state tracking module 118 enables comprehensive tracking and visualization of environmental conditions such as radiation exposure, allowing actors in the environment to become aware of actions during procedures that increase their overall exposure and dose accumulation through the extended reality system 102, enabling real-time adjustment of positioning and procedures to minimize radiation exposure while maintaining procedural effectiveness. The temporal data storage module 114 can maintain historical radiation exposure records for each individual, enabling tracking of cumulative doses over multiple procedures.

The clinical workflow engine 122 can provide the system 100 with the ability to define, execute, and navigate through structured sequences of clinical tasks that collectively constitute a complete medical procedure. A clinical workflow can be an ordered collection of clinical tasks necessary to complete a procedure, with user control over transitions among tasks including when to transition and the direction of transition either sequentially to a later task, or back to an earlier task. Each clinical task defines an overall system state that includes the configuration and state of each system in the suite and the holographic user interface for each operator, including displays and tools. When the clinical workflow engine 122 transitions from one task to the next, the system 100 can automatically reconfigure the integrated medical systems 106 and each operator's virtual display environment to reflect the states and interfaces appropriate to the new task. This approach reduces the burden on individual operators to manually reconfigure their displays and equipment as the procedure advances. An advantage of implementing structured clinical workflows is that they establish consistency and uniformity for physicians and institutions, enabling implementation of best practices across procedures.

An example clinical workflow can include the following sequential stages: Start, Access, Navigation, Diagnosis, Treatment Planning, Treatment, Treatment Assessment, Withdraw Devices, Closure, and End. Each stage can define the specific systems and interfaces that each operator requires to perform their role during that phase of the procedure. The primary physician's tasks can include inserting vascular access, navigating devices to the heart under X-ray image guidance, acquiring diagnostic imaging and FFR measurements, obtaining IVUS imaging, performing angioplasty and stent deployment under image guidance, and removing devices under X-ray guidance. Simultaneously, the radiology technician can monitor X-ray image quality and record key images, the anesthesiologist can monitor patient vitals and maintain patient sedation, and the nurse can monitor patient physiology and administer medications.

Any operator can initiate a procedure by gathering a list of scheduled procedures from the scheduling system, claiming an existing procedure, or creating a new procedure and entering patient and procedure data. Upon procedure start, the system 100 can communicate that patient and procedure data to all integrated medical systems 106 simultaneously. The procedure initiation flow can include a patient timeout step and can accommodate both known and unknown procedure types, with the clinical workflow engine 122 configuring the appropriate clinical task sequence for the selected procedure.

Users can control transitions among workflow tasks, including the direction of transition, for example, advancing sequentially to the next task, skipping forward to a later task, or returning to an earlier task as clinical circumstances require. The clinical workflow engine 122 does not restrict task transitions to a strictly linear sequence; rather, it provides a structured framework within which users retain the flexibility to navigate the workflow as needed to accommodate intraoperative developments. The state tracking module 118 enables these role-specific workflows by maintaining awareness of what information each user needs at different procedure stages, working in conjunction with the clinical workflow engine 122 to automatically update system configurations and operator interfaces as task transitions occur.

The clinical workflow engine 122 can operate in coordination with the role-based access control module 116, state tracking module 118, and temporal data storage module 114 throughout procedure execution. Key events tracked by the temporal data storage module 114 can include procedure start and end times, medication administration, treatment delivery, analysis results, and user-defined events for tracking procedure execution and workflow efficiencies. Through integration with hospital information and billing systems, the clinical workflow engine 122 can support post-procedure reporting and documentation workflows in addition to intra-procedure task management.

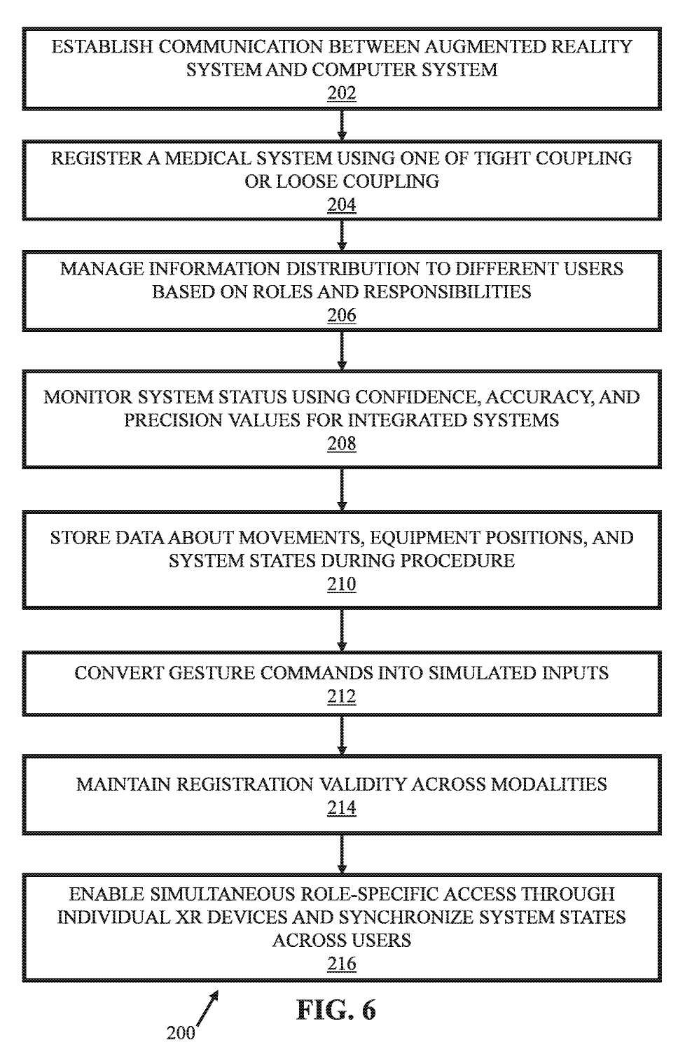

With reference to FIG. 6, a method 200 for multi-user extended reality integration and coordinated control of medical systems can be implemented through the system 100 described herein. The method 200 can include a step 202 of establishing communication between the extended reality system 102 and the computer system 104 through the network 101, enabling data exchange between system components and forming the communication foundation through which the remaining steps of the method 200 operate.

The method 200 can include a step 204 of registering one or more medical systems 106 through the registration management module 112 using one of a tight coupling or loose coupling methodology selected based on the technical capabilities of each medical system 106. Where a medical system 106 exposes a native data interface, the system integration module 110 employs tight coupling to enable direct data interchange over dedicated wired, wireless, or Bluetooth interfaces at lower bandwidth, lower latency, and higher fidelity. Where a medical system 106 does not expose a native interface, the system integration module 110 employs loose coupling through video capture and simulated control inputs. The registration management module 112 enables the system 100 to understand the unique data space and coordinate system of each medical system 106 and perform data space transformations that allow information from each system to be displayed in a unified extended reality environment.

The method 200 can include a step 206 of managing information distribution to different users based on their roles and responsibilities through the role-based access control module 116, ensuring that each operator receives the data and system control access appropriate to their role in the procedure. The method 200 can further include a step 208 of monitoring system configurations and operational status through the state tracking module 118, using specific confidence values, accuracy values, and precision values for each integrated medical system 106, to maintain continuous awareness of equipment states and operational parameters throughout the procedure.

The method 200 can include a step 210 of storing temporal data through the temporal data storage module 114, including movements, equipment positions, recorded data elements such as images and signals, system states, and key events, enabling healthcare providers to later recall and compare information from any prior procedural time point while maintaining contextual system state information and registration validity.

The method 200 can include a step 212 of converting gesture commands on displayed virtual panels 124 into simulated inputs for loosely coupled medical systems 106, enabling users to control legacy and third-party systems through emulated mouse cursor positions, clicks, and keyboard inputs, including through the virtual keyboard 126, without requiring a native integration interface from those systems.

The method 200 can include a step 214 of maintaining registration validity across different modalities while tracking equipment and patient movements through the registration management module 112. As the patient, patient table, or imaging equipment moves during a procedure, the registration management module 112 can apply registration transformations to preserve spatial relationships of acquired data relative to patient anatomy, storing and synchronizing temporal data about movements across different system time references for tightly integrated systems. The registration management module 112 can maintain system-specific confidence values, accuracy values, and precision values for each integrated medical system 106 to enable the system 100 to display and align information from multiple sources in the extended reality environment while preserving spatial relationships, regardless of how each individual medical system 106 handles registration.

The method 200 can include a step 216 of enabling multiple medical professionals to simultaneously access role-specific information through individual Xr devices 108 while synchronizing system states across users through the coordinated operation of the role-based access control module 116, state tracking module 118, and temporal data storage module 114. The system 100 can maintain temporal and spatial reference systems shared between users through data exchange channels, synchronizing virtual content positioning, user interface element values, selected frame identifiers, and display settings across all connected users. When changes occur to system states or virtual content positioning, these updates can be propagated across all connected users while maintaining appropriate role-based access controls and preserving registration validity throughout the procedure.

### Examples

The following examples illustrate the system 100 operating as an integrated whole across representative clinical scenarios. Each example demonstrates how the extended reality system 102, computer system 104, and integrated medical systems 106 function in coordination with the integration module 110, the registration management module 112, temporal data storage module 114, role-based access control module 116, state tracking module 118, clinical workflow engine 122, and hazard monitoring module 120 working together to support procedure execution, multi-user coordination, and clinical decision-making. These examples are illustrative of particular embodiments and are not intended to limit the scope of the invention.

### Example 1: Percutaneous Coronary Intervention PTCA

A scrub technician initiates the procedure by claiming the scheduled PTCA from the scheduling system and entering patient and procedure data. Upon procedure start, the system 100 communicates that data to all integrated medical systems 106 simultaneously, including the X-ray angiography system, patient monitoring equipment, hemodynamics system, and IVUS system, and the clinical workflow engine 122 configures the appropriate clinical task sequence following a patient timeout step. The role-based access control module 116 and state tracking module 118 then configure a customized holographic interface for each operator through their individual XR devices 108. The primary physician receives active fluoroscopic imaging displays, imaging geometry controls, and tracking system overlays. The anesthesiologist receives hemodynamic monitoring data and a medication administration record. The nurse receives patient vitals and access to inventory management. The radiology technician receives image quality monitoring tools and key image capture controls. Each operator's display is positioned ergonomically within their field of view, enabling each team member to access the information they need without competing for a shared central display.

As the clinical workflow engine 122 advances to the Navigation stage, the primary physician navigates devices to the heart under X-ray image guidance. The registration management module 112 maintains the spatial alignment of the C-arm imaging geometry in real-time within the extended reality environment, automatically adjusting the position of displayed images as the C-arm moves. Through tight coupling with the X-ray angiography system, the system 100 tracks C-arm and patient table movements, fixing acquired images relative to the patient so that they move appropriately in the virtual space as the table is repositioned. Upon advancing to the Diagnosis stage, the primary physician acquires diagnostic DSA imaging, performs FFR measurements, and obtains IVUS imaging, while the radiology technician simultaneously monitors image quality and records key images, the anesthesiologist monitors patient vitals and maintains sedation, and the nurse monitors physiology and administers medications, all through their respective role-specific interfaces without disrupting one another.

During the Treatment Planning stage, the primary physician identifies treatable lesions and makes treatment decisions, after which the clinical workflow engine 122 advances to the Treatment stage and the physician performs angioplasty and deploys a stent under image guidance. Throughout this stage, the hazard monitoring module 120 continuously tracks radiation exposure for all operators, displaying real-time holographic color overlays showing dose levels and alerting operators who move into areas exceeding configured thresholds. The temporal data storage module 114 records procedure execution data throughout, including image acquisitions, treatment delivery events, equipment states, and medication administrations, with associated timestamps.

Upon advancing to the Treatment Assessment stage, the primary physician acquires post-treatment DSA imaging, performs post-treatment FFR measurements, and obtains follow-up IVUS imaging, with the system 100 enabling direct comparison of pre- and post-treatment imaging while maintaining registration validity and spatial relationships across imaging sessions. The clinical workflow engine 122 then advances through the Withdraw Devices, Closure, and End stages, during which the physician removes devices under X-ray guidance, removes the vascular access port, and seals the vessel, with the clinical workflow engine 122 supporting post-procedure documentation and reporting workflows through integration with hospital information and billing systems upon procedure end.

### Example 2: Multi-Session Surgical Planning and Intraoperative Navigation

In the days prior to the procedure, a technician accesses the integrated advanced imaging workstation through the extended reality system 102 to perform image analysis and procedure planning. Through tight coupling with the workstation, the technician manipulates and processes previously acquired DICOM imaging data while the primary physician simultaneously reviews the same data through their own XR device 108 and provides input and approval through a view-only interface managed by the role-based access control module 116. During this session, the team generates derived planning data including detailed anatomical models, target location markers, tumor visualizations, and procedural exclusion zones. The temporal data storage module 114 preserves all planning activities including which users performed specific analyses and made key decisions enabling the team to refine the plan across multiple sessions while maintaining registration validity of all derived data.

On the day of the procedure, the registration management module 112 registers the patient's current anatomy to the pre-operative imaging data using a combination of surface registration and tracked ultrasound. The system 100 detects the patient's current position using LIDAR scanning capabilities, analyzing surface contours and applying statistical shape modeling to automatically identify anatomical landmarks without requiring manual placement of fiducial markers. The state tracking module 118 assigns confidence values to the resulting registration, and the system 100 displays the registered pre-operative planning data, including the anatomical model, target markers, and exclusion zones, as holographic overlays registered directly to the patient's anatomy in the extended reality environment.

As the procedure advances, the system 100 maintains registration validity by tracking patient and patient table movements through optical tracking systems integrated with the extended reality system 102. When the patient table moves, the registration management module 112 automatically adjusts the positioning of all virtual content to maintain proper spatial relationships with the patient's anatomy. The primary physician navigates to the target using the registered holographic overlays while the technician monitors imaging data through their XR device 108, with the state tracking module 118 continuously updating confidence values for the active registration and the temporal data storage module 114 recording all positional and imaging data with timestamps, enabling the team to review and compare information from any point during the procedure while maintaining full contextual system state information.

Midway through the procedure, a remote consulting specialist joins through a virtual reality implementation that presents the procedure room environment and relevant medical data, enabling the specialist to view the registered holographic overlays, live imaging feeds, and patient monitoring data in context and communicate with the operating team through a combination of voice, video, and text. The role-based access control module 116 limits the remote participant to a passive view-only role, ensuring they can observe and provide input without having control over virtual content or system settings.

### Example 3: Multi-User Coordination with Remote Participation

At procedure start, the system 100 configures simultaneous participation for a procedure team comprising a primary physician, a fellow, a radiology technician, an anesthesiologist, a nurse, and a remote collaborator; each with a customized user interface and role-specific defaults established through the role-based access control module 116 and state tracking module 118. The primary physician's XR device 108 displays live imaging data registered to patient anatomy, treatment system controls, and tracking overlays. The anesthesiologist's XR device 108 displays hemodynamics data, ECG signals, medication administration records, and respiratory monitoring through tight coupling with the patient monitoring system. The nurse's XR device 108 displays patient vitals, inventory management, and medication administration data through a loosely coupled integration, with gesture-based interaction on virtual display panels that convert to simulated mouse inputs on the underlying system.

At a key moment during the procedure, the primary physician pushes a fluoroscopic image to every team member's XR device 108 simultaneously to direct attention to a clinical finding. The role-based access control module 116 continues to enforce access restrictions appropriate to each role in parallel: the technician retains full control over imaging system parameters while the nurse retains view-only access to imaging output and full control over medication and inventory systems, with some information common to all participants and other information presented only to the operators for whom it is relevant.

The fellow's participation illustrates how the system 100 supports concurrent training without disrupting procedure flow. The fellow can view all physician displays and access planning data, but control permissions are limited by the role-based access control module 116 to prevent inadvertent system interactions, and the fellow's XR device 108 is configured to display educational annotations and guidance overlays not visible to the primary physician. The remote collaborator connects through a virtual reality implementation and receives the display information of any connected system in the suite, including the procedure room environment and registered medical data, communicating with the team through a combination of voice, video, and text. System state synchronization ensures that virtual objects remain properly positioned relative to physical space across all connected users, with changes to system states and virtual content synchronized across local and remote participants while maintaining appropriate role-based access controls.

### Example 4: Equipment Repositioning and Pre/Post-Treatment Imaging Comparison

During the imaging phase of the procedure, the primary physician positions the patient table and C-arm to obtain optimized visualization of the target anatomical region and acquires pre-treatment images. The system 100 stores this geometric configuration through the temporal data storage module 114 including table position, C-arm angle, and imaging parameters, with the state tracking module 118 recording specific confidence, accuracy, and precision values for all equipment positions. Through tight coupling with the X-ray angiography system, the registration management module 112 fix the acquired pre-treatment images relative to the patient, so that those images move appropriately in the virtual space with any subsequent patient table movement.

The physician then repositions the patient table and imaging geometry to optimize visualization of a different anatomical region for treatment delivery, with this second configuration also stored through the temporal data storage module 114. After performing treatment at the second position, the system 100 enables the physician to review pre-treatment imaging from the first position while maintaining proper spatial relationships adjusted for the intervening table and imaging geometry changes allowing direct comparison of pre-treatment anatomy to the current post-treatment state without returning equipment to the original position.

When post-treatment imaging at the initial position is desired, the system 100 automatically restores equipment positions through direct control interfaces, communicating with the patient table control systems to drive table movement and with the imaging systems to adjust imaging geometry to precisely match the stored positional state, without requiring the physician to manually recreate the original setup. With equipment returned to the initial configuration, the physician acquires post-treatment images that are directly comparable to the pre-treatment images stored earlier in the procedure. The temporal data storage module 114 preserves the full context of system configurations at each imaging position, and the state tracking module 118 maintains registration validity throughout all positional transitions, ensuring that images from different time points and equipment positions can be meaningfully compared within the extended reality environment.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A system for multi-user extended reality integration of medical systems, comprising:
an extended reality system defining an extended reality environment, including a plurality of extended reality (XR) devices, each XR device configured to selectively display an augmented representation to a respective user;
a computer system in communication with the extended reality system, the computer system configured to integrate with a plurality of medical systems, to selectively couple to each medical system through one of a tight coupling or a loose coupling, to maintain spatial registration between data from each integrated medical system and at least one of a suite coordinate space representing the physical procedure room and a patient coordinate space.

2. The system of claim 1, wherein the tight coupling comprises direct communication with each medical system over an interface selected from the group consisting of a dedicated wired interface, a wireless interface, and a Bluetooth interface.

3. The system of claim 2, wherein the tight coupling enables direct interchange of data selected from the group consisting of two-dimensional images, three-dimensional images, time-varying signal data, system state information, and report data.

4. The system of claim 1, wherein the loose coupling comprises capturing video output from a medical system and replicating that video onto one or more virtual panels within the extended reality environment.

5. The system of claim 4, wherein user gesture commands on the virtual panels are converted into at least one of emulated mouse cursor positions and emulated mouse clicks for controlling the medical system.

6. The system of claim 1, wherein the computer system is further configured to configure each XR device to display role-specific information based on a role associated with the respective user.

7. The system of claim 1, wherein the computer system comprises a registration management module configured to maintain a suite coordinate space representing the physical procedure room.

8. The system of claim 7, wherein the registration management module is further configured to maintain a patient coordinate space defining a spatial relationship between acquired imaging data and a patient's physical anatomy.

9. The system of claim 8, wherein the registration management module is further configured to maintain a system-specific coordinate space for each integrated medical system.

10. The system of claim 9, wherein the registration management module is further configured to perform coordinate transformations enabling data from any integrated medical system to be displayed in any coordinate space to which that system is registered.

11. The system of claim 7, wherein the registration management module is further configured to track patient and patient table movements and to automatically adjust positioning of virtual content within the extended reality environment to maintain spatial registration with the patient's anatomy as movement occurs.

12. The system of claim 1, wherein the computer system further comprises a temporal data storage module configured to continuously record procedure execution data throughout a medical procedure, the procedure execution data comprising at least one of user positions and interactions, acquired images and signals, and system states selected from the group consisting of imaging geometry, patient table geometry, and combinations thereof.

13. The system of claim 1, wherein the computer system further comprises a role-based access control module configured to filter content displayed through each XR device and a level of system control available to each user based on a role assigned to the respective user, and to support active participants having control over virtual content and passive participants having view-only access.

14. The system of claim 13, wherein the role-based access control module is further configured to synchronize shared information across all connected users while maintaining role-specific access restrictions, such that information common to all operators is displayed simultaneously across all XR devices and information specific to a particular role is displayed only to the operator assigned that role.

15. The system of claim 13, wherein the computer system further comprises a state tracking module configured to monitor configurations and operational status of all integrated medical systems throughout a medical procedure using system-specific confidence values, accuracy values, and precision values.

16. The system of claim 15, wherein the state tracking module is further configured to coordinate with the role-based access control module to update each display environment of each user as the procedure advances through clinical stages.

17. A method for multi-user extended reality integration of medical systems, comprising:
providing an extended reality environment through a plurality of extended reality (XR) devices, each XR device selectively displaying an augmented representation to a respective user;
integrating with a plurality of medical systems;
selectively coupling to each medical system through one of a tight coupling or a loose coupling; and
maintaining spatial registration between data from each integrated medical system and at least one of a suite coordinate space representing a physical procedure room and a patient coordinate space.

18. The method of claim 17, wherein selectively coupling comprises communicating directly with each medical system over an interface selected from the group consisting of a dedicated wired interface, a wireless interface, and a Bluetooth interface.

19. The method of claim 17, wherein selectively coupling comprises capturing video output from a medical system and replicating that video onto one or more virtual panels within the extended reality environment.

20. The method of claim 17, further comprising continuously recording procedure execution data throughout a medical procedure, the procedure execution data comprising at least one of user positions and interactions, acquired images and signals, and system states selected from the group consisting of imaging geometry, patient table geometry, and combinations thereof.
